(19) United States
(12) Patent Application Publication
(10) Pub. No.: US 2007/0185186 A1
(43) Pub. Date: Aug. 9, 2007

(54) COMPOSITIONS FOR THE DELIVERY OF SUBSTITUTED NAPTHYL INDOLE DERIVATIVES AND METHODS OF THEIR USE

(75) Inventors: Behnam Abdolalizadeh, Congers, NY (US); T. Ian Armstrong, Bishops Waltham (GB); James Provost, Waltham Chase (GB); Zerina B. Shafi, Fareham (GB); M. Sherry Ku, Thiells, NY (US)

Correspondence Address: WOODCOCK WASHBURN LLP/WYETH, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/669,810

(22) Filed: Jan. 31, 2007

Related U.S. Application Data
(60) Provisional application No. 60/765,122, filed on Feb. 3, 2006.

(51) Int. Cl. A61K 31/404; C07D 209/04

(52) U.S. Cl. 514/412; 540/490

(57) ABSTRACT

This invention relates to compositions for the delivery of substituted naphthyl indole derivatives as well as to the use of these compositions and methods for treating disease.

### CROSS-REFERENCE TO RELATED APPLICATIONS

This application claims the benefit of U.S. Provisional Application No. 60/765,122 filed Feb. 3, 2006, the entire disclosure of which is incorporated herein by reference.

### FIELD

This invention relates, inter alia, to compositions for the delivery of substituted naphthyl indole derivatives as well as to the use of these compositions and methods for treating disease.

### BACKGROUND

Plasminogen activator inhibitor-1 (PAI-1) is a major regulatory component of the plasminogen-plasmin system. PAI-1 is the principal physiologic inhibitor of both tissue type plasminogen activator (t-PA) and urokinase type plasminogen activator (u-PA). PAI-1 is found at low levels (5-10 ng/ml) in the plasma of healthy individuals, but is elevated significantly in a number of disease states, including atherosclerosis, deep vein thrombosis, and non-insulin dependent diabetes mellitus. PAI-1 stabilizes both aerial and venous thrombi, contributing respectively to coronary artery occlusion in post myocardial infarction and venous thrombi following postoperative recovery from orthopedic surgery. Elevated plasma levels of PAI-1 have been associated with thrombotic events as indicated by animal experiments (Krishnamurti, Blood, 69, 798 (1987); Reilly, Arteriosclerosis and Thrombosis, 11, 1276 (1991); Carmeliet, Journal of Clinical Investigation, 92, 2756 (1993)) and clinical studies (Rocha, Fibrinolysis, 8, 294, 1994; Aznar, Haemostasis 24, 243 (1994)). Antibody neutralization of PAI-1 activity resulted in promotion of endogenous thrombolysis and reperfusion (Biemond, Circulation, 91, 1175 (1995); Levi, Circulation 85, 305, (1992)). Elevated levels of PAI-1 have also been implicated in many other diseases, including diseases of women such as polycystic ovary syndrome (Nordt, Journal of clinical Endocrinology and Metabolism, 85, 4, 1563 (2000)) and bone loss induced by estrogen deficiency (Daci, Journal of Bone and Mineral Research, 15, 8, 1510 (2000)). Compounds possessing PAI-1 inhibitory activity are useful for the treatment of a wide variety of conditions originating from fibrinolytic disorders.

U.S. Pat. No. 6,800,654, incorporated herein by reference in its entirety and for all purposes discloses compounds of Formula I that inhibit PAI-1 activity: 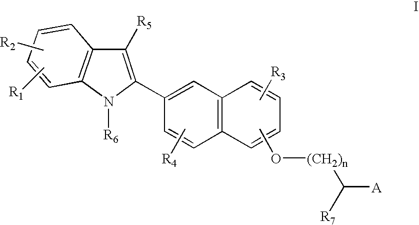
wherein R₁, R₂, R₃, R₄, R₅, R₆, R₇, n, and A are as defined herein. A need exists for pharmaceutical compositions and processes for the effective delivery of compounds of Formula I and/or pharmaceutically acceptable salts thereof to the GI tract. The present invention is directed to this and other important uses.

### BRIEF DESCRIPTION OF THE DRAWINGS

FIG. 1 provides a graph showing the mean plasma levels in dogs following administration of a composition of the present invention. 3 capsules comprising 25 mg of the active ingredient 1-Benzyl-3-pentyl-2-[6-(1H-tetrazol-5-ylmethoxy)-2-naphthyl]-1H-indol administered to the dogs in fasted and fed conditions.

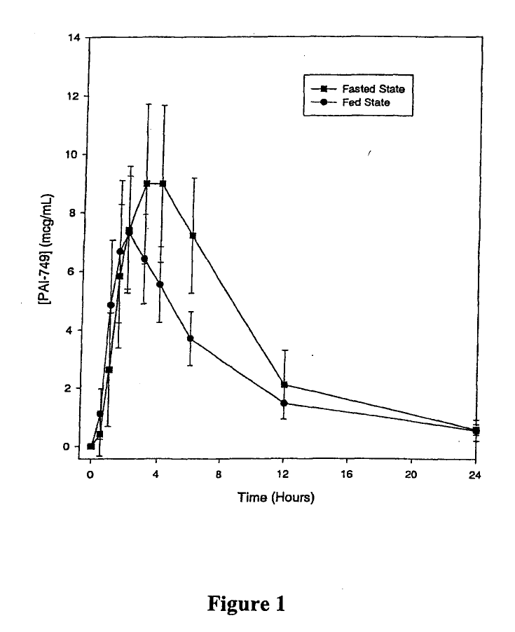

### SUMMARY

The present invention provides, inter alia, compositions comprising one or more compounds of Formula I and/or pharmaceutically acceptable salts or ester forms thereof, in combination with one or more surfactants. The compositions are preferably formulated for delivery to a subject for the treatment of various diseases and disorders. The compositions can be used, for example, to inhibit the serine protease inhibitor PAI-1, and to treat or prevent diseases and conditions associated with the production and/or action of PAI-1. These diseases and conditions include, for example, noninsulin dependent diabetes mellitus; cardiovascular disease caused by noninsulin dependent diabetes mellitus; thrombosis, including, but not limited to venous thrombosis, arterial thrombosis, and deep vein thrombosis; formation of atherosclerotic plaques; myocardial ischemia; atrial fibrillation; coagulation syndromes; pulmonary thrombosis; cerebral thrombosis; thromboembolic complications of surgery (such as joint replacement); peripheral arterial occlusion; stroke; including, but not limited to, stroke associated with or resulting from atrial fibrillation; and other diseases and conditions recited herein.

A compound of Formula I is as shown below:

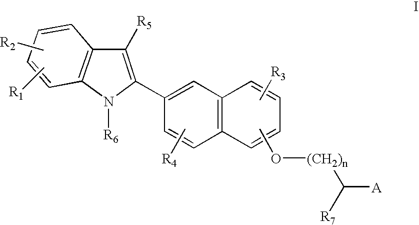

wherein:

R₁, R₂, R₃, and R₄ are each, independently, one or more groups selected from hydrogen, alkyl, cycloalkyl, —CH₂-cycloalkyl, alkanoyl, halogen, hydroxy, optionally substituted aryl, perfluoroalkyl, alkoxy, amino, alkylamino of 1-6 carbons, dialkylamino, or perfluoroalkoxy;

R₅ is hydrogen, alkyl of 1-6 carbons, perfluoroalkyl, optionally substituted aryl, alkanoyl, or aroyl;

R₆ is hydrogen, alkyl, alkylaryl, optionally substituted benzyl, alkanoyl, or aroyl;

R₇ is hydrogen, alkyl, alkylaryl, or optionally substituted aryl;

n is an integer of 0-6;

A is COOH, or an acid mimic;

or a pharmaceutically acceptable salt or ester form thereof.

In certain embodiments, the compositions of the present invention comprise about 1% to about 90% by weight of an active ingredient of Formula I and about 0.1% to about 10% by weight of a surfactant.

In some embodiments, the ratio of active ingredient to surfactant is about 5:1.

In some embodiments, the surfactant is an alkyl sulfate, a polyoxyalkylene sorbitan ester, or a poloxamer. In certain aspects, the surfactant is sodium lauryl sulfate.

In some embodiments, a composition of the present invention further comprises a disintegrant. In some aspects, the disintegrant is carboxymethyl cellulose or a pharmaceutically acceptable basic salt form thereof; starch glycolate or a pharmaceutically acceptable basic salt form thereof; or mixtures thereof. In some aspects, the disintegrant is the sodium salt of cross-linked carboxymethyl cellulose and the starch glycolate is the sodium salt of starch glycolate.

In some embodiments, a composition of the present invention further comprises a glidant. In some aspects, the glidant is silicon dioxide, silica gel, talc or mixtures thereof. In some aspects, the silicon dioxide is colloidal.

In some embodiments, a composition of the present invention further comprises a lubricant. In some aspects the lubricant is magnesium stearate, stearic acid, talc, sodium stearyl fumarate, or mixtures thereof.

In some embodiments, a composition of the present invention further comprises a filler. In some aspects, the filler is microcrystalline cellulose, lactose, calcium carbonate, calcium phosphate, maltodextrin, dextrose, sucrose, fructose, maltose, mannitol, starch, or mixtures thereof. In some aspects, the filler is microcrystalline cellulose.

In some embodiments, the compound of Formula I is 1-Benzyl-3-pentyl-2-[6-(1H-tetrazol-5-ylmethoxy)-2-naphthyl]-1H-indole or a pharmaceutically acceptable salt or ester form thereof. In some embodiments, the compound of Formula I is 1-Benzyl-3-pentyl-2-[6-(1H-tetrazol-5-ylmethoxy)-2-naphthyl]-1H-indole.

In some embodiments, a composition of the present invention comprises from about 1% to about 2% by weight of a compound of Formula I, and further comprises about 3% to about 20% by weight of a disintegrant and about 0.1% to about 5% by weight of a glidant. In certain aspects, the disintegrant is carboxymethyl cellulose; starch glycolate; or a pharmaceutically acceptable basic salt form thereof; and the glidant is silicon dioxide or silica gel.

In some embodiments, a composition of the present invention comprises from about 10% to about 20% by weight of a compound of Formula I, and further comprises about 3% to about 20% by weight of a disintegant and about 0.1% to about 5% by weight of a glidant. In certain aspects, the disintegrant is carboxymethyl cellulose; starch glycolate; or a pharmaceutically acceptable basic salt form thereof; and the glidant is silicon dioxide or silica gel.

In some embodiments, the composition is formulated as an oral dosage form, preferably in the form of a capsule. In certain aspects, the capsule is coated. In certain embodiments, the capsule is a hydroxypropylmethyl cellulose capsule.

In some embodiments, the dosage form is in the form of a dry blend.

The present invention also provides, inter alia, processes for preparing a compound of Formula I comprising mixing a compound of Formula I with at least one surfactant; at least one disintegrant; and at least one glidant thereby forming a mixture blend thereof.

The present invention also provides, inter alia, products produced by the process of dry blending a composition of the present invention.

### DETAILED DESCRIPTION

The present invention provides, inter alia, compositions, including oral dosage forms, comprising one or more compounds of Formula I and/or pharmaceutically acceptable salts and ester forms thereof, in combination with one or more surfactants. A compound of Formula I is as shown below wherein:

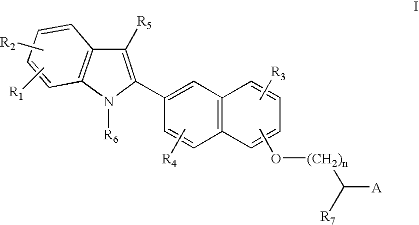

wherein:

R₁, R₂, R₃, and R₄ are each, independently, one or more groups selected from hydrogen, alkyl, cycloalkyl, —CH₂-cycloalkyl, alkanoyl, halogen, hydroxy, optionally substituted aryl, perfluoroalkyl, alkoxy, amino, alkylamino of 1-6 carbons, dialkylamino, or perfluoroalkoxy;

R₅ is hydrogen, alkyl of 1-6 carbons, perfluoroalkyl, optionally substituted aryl, alkanoyl, or aroyl;

R₆ is hydrogen, alkyl, alkylaryl, optionally substituted benzyl, alkanoyl, or aroyl;

R₇ is hydrogen, alkyl, alkylaryl, or optionally substituted aryl;

n is an integer of 0-6;

A is COOH, or an acid mimic;

or a pharmaceutically acceptable salt or ester form thereof.

In certain embodiments,

R₁, R₂, R₃, and R₄ are each, independently, hydrogen, alkyl of 1-6 carbons, cycloalkyl of 3-5 carbon atoms, —CH₂-cycloalkyl of 3-5 carbon atoms, alkanoyl of 1-6 carbons, halogen, hydroxy, aryl optionally substituted with from 1 to 3 groups selected from R₈, perfluoroalkyl of 1-6 carbons, alkoxy of 1-6 carbons, amino, alkylamino of 1-6 carbons, dialkylamino of 1-6 carbons, or perfluoroalkoxy of 1-6 carbons;

R₅ is hydrogen, alkyl of 1-6 carbons, perfluoroalkyl of 1-6 carbons, aryl substituted with R₈, alkanoyl of 1-6 carbons, or aroyl optionally substituted with from 1 to 3 groups selected from R₈;

R₆ is hydrogen, alkyl of 1-6 carbons, alkylaryl, benzyl substituted with R₈, alkanoyl of 1-6 carbons, or aroyl optionally substituted with from 1 to 3 groups selected from R₈;

R₇ is hydrogen, alkyl of 1-6 carbons, alkylaryl, or aryl optionally substituted with from 1 to 3 groups selected from R₈;

n is an integer of 0-6;

A is COOH, or an acid mimic; and

R₈ is hydrogen, alkyl of 1-6 carbons, cycloalkyl of 3-5 carbons, —CH₂-cycloalkyl of 3-5 carbon atoms, alkanoyl of 1-6 carbons, halogen, hydroxy, perfluoroalkyl of 1-6 carbons, alkoxy of 1-6 carbons, amino, alkylamino of 1-6 carbons, dialkylamino of 1-6 carbons, or perfluoroalkoxy of 1-6 carbons; or a pharmaceutically acceptable salt or ester form thereof.

In certain embodiments,

R₁, R₂, R₃, and R₄ are each, independently, hydrogen, alkyl of 1-3 carbons, cycloalkyl of 3-5 carbon atoms, —CH₂-cycloalkyl of 3-5 carbon atoms, alkanoyl of 1-3 carbons, halogen, hydroxy, aryl optionally substituted with from 1 to 3 groups selected from R₈, perfluoroalkyl of 1-3 carbons, alkoxy of 1-3 carbons, amino, alkylamino of 1-3 carbons, dialkylamino of 1-3 carbons, or perfluoroalkoxy of 1-3 carbons;

R₅ is hydrogen, alkyl of 1-6 carbons, perfluoroalkyl of 1-6 carbons, aryl substituted with R₈, alkanoyl of 1-6 carbons, or aroyl optionally substituted with from 1 to 3 groups selected from R₈;

R₆ is hydrogen, alkyl of 1-6 carbons, alkylaryl, benzyl substituted with R₈, alkanoyl of 1-6 carbons, or aroyl optionally substituted with from 1 to 3 groups selected from R₈;

R₇ is hydrogen, alkyl of 1-6 carbons, alkylaryl, or aryl optionally substituted with from 1 to 3 groups selected from R₈;

n is an integer of 0-6;

A is COOH, or an acid mimic; and

R₈ is hydrogen, alkyl of 1-6 carbons, cycloalkyl of 3-5 carbons, —CH₂-cycloalkyl of 3-5 carbon atoms, alkanoyl of 1-3 carbons, halogen, hydroxy, perfluoroalkyl of 1-3 carbons, alkoxy of 1-3 carbons, amino, alkylamino of 1-3 carbons, dialkylamino of 1-3 carbons, or perfluoroalkoxy of 1-3 carbons; or a pharmaceutically acceptable salt or ester form thereof.

In certain embodiments,

R₁, R₂, R₃, and R₄ are each, independently, hydrogen, alkyl of 1-3 carbons, cycloalkyl of 3-5 carbon atoms, —CH₂-cycloalkyl of 3-5 carbon atoms, alkanoyl of 1-3 carbons, halogen, hydroxy, aryl optionally substituted with from 1 to 3 groups selected from R₈, perfluoroalkyl of 1-3 carbons, alkoxy of 1-3 carbons, amino, alkylamino of 1-3 carbons, dialkylamino of 1-3 carbons, or perfluoroalkoxy of 1-3 carbons;

R₅ is hydrogen, alkyl of 1-6 carbons, perfluoroalkyl of 1-6 carbons, aryl substituted with R₈, alkanoyl of 1-6 carbons, or aroyl optionally substituted with from 1 to 3 groups selected from R₈;

R₆ is hydrogen, alkyl of 1-6 carbons, alkylaryl, benzyl substituted with R₈, alkanoyl of 1-6 carbons, or aroyl optionally substituted with from 1 to 3 groups selected from R₈;

R₇ is hydrogen, alkyl of 1-6 carbons, alkylaryl, or aryl optionally substituted with from 1 to 3 groups selected from R₈;

n is an integer of 0-6;

A is COOH, or an acid mimic; and

R₈ is hydrogen, alkyl of 1-3 carbons, cycloalkyl of 3-5 carbons, —CH₂-cycloalkyl of 3-5 carbon atoms, alkanoyl of 1-3 carbons, halogen, hydroxy, perfluoroalkyl of 1-3 carbons, alkoxy of 1-3 carbons, amino, alkylamino of 1-3 carbons, dialkylamino of 1-3 carbons, or perfluoroalkoxy of 1-3 carbons; or a pharmaceutically acceptable salt or ester form thereof.

In certain exemplary embodiments, R₄ is hydrogen, alkyl of 1 to 6 carbon atoms, perfluoroalkyl of 1 to 6 carbon atoms, aryl substituted with R₈, alkanoyl of 1 to 6 carbon atoms, or aroyl optionally substituted with from 1 to 3 groups independently selected from R₈.

In certain exemplary embodiments, R₁ is hydrogen. In certain exemplary embodiments, R₂ is hydrogen. In certain exemplary embodiments, R₃ is bromine or hydrogen. In certain exemplary embodiments, R₄ is hydrogen. In certain exemplary embodiments, R₅ is pentyl. In certain exemplary embodiments, R₆ is benzyl, methyl, acyl, (2-trifluoromethyl)benzyl and (4-tert-butyl)benzyl. In certain exemplary embodiments, R₇ is hydrogen. In certain exemplary embodiments, n is zero. In certain exemplary embodiments, A is CO₂H or tetrazole.

The present invention also provides processes for making such compositions and methods of administering them to a mammal, e.g., human subject.

Exemplary compositions of the present invention can be used to inhibit the serine protease inhibitor PAI-1, and are therefore useful in the treatment or prophylaxis of those processes which involve the production and/or action of PAI-1. Thus, exemplary compositions of the invention are useful in the treatment or prevention of noninsulin dependent diabetes mellitus and cardiovascular disease caused by such condition, and treatment and prevention of thrombotic events associated with coronary artery and cerebrovascular disease. Exemplary compositions are also useful for inhibiting the disease process involving the thrombotic and prothrombotic states which include, but are not limited to, formation of atherosclerotic plaques, venous and arterial thrombosis, myocardial ischemia, atrial fibrillation, deep vein thrombosis, coagulation syndromes, pulmonary thrombosis, cerebral thrombosis, thromboembolic complications of surgery (such as joint replacement), and peripheral arterial occlusion. Exemplary pharmaceutical compositions are also useful in treating or preventing stroke associated with or resulting from atrial fibrillation.

Exemplary compositions can also be used in the treatment or prevention of diseases associated with extracellular matrix accumulation, including, but not limited to, renal fibrosis, chronic obstructive pulmonary disease, polycystic ovary syndrome, restenosis, renovascular disease and organ transplant rejection.

Exemplary compositions of the invention can also be used in the treatment or prevention of malignancies, and diseases associated with neoangiogenesis (such as diabetic retinopathy).

Exemplary compositions can also be used in conjunction with and following processes or procedures involving maintaining blood vessel patency, including vascular surgery, vascular graft and stent patency, organ, tissue and cell implantation and transplantation.

Exemplary compositions can also be used in the treatment of Alzheimer's disease. This method can also be characterized as the inhibition of plasminogen activator by PAI-1 in a mammal, particularly a human, experiencing or subject to Alzhemier's disease. This method can also be characterized as a method of increasing or normalizing levels of plasmin concentration in a mammal, particularly those experiencing or subject to Alzheimer's disease.

Exemplary compositions can be used for the treatment or prevention of myelofibrosis with myeloid metaplasia by regulating stromal cell hyperplasia and increases in extracellular matrix proteins.

Exemplary compositions can also be used in conjunction with protease inhibitor-containing highly active antiretroviral therapy (HAART) for the treatment or prevention of diseases which orginate from fibrinolytic impairment and hyper-coagulability of HIV-1 infected patients receiving such therapy.

Exemplary compositions can be used for the treatment or prevention of diabetic nephropathy and renal dialysis associated with nephropathy.

Exemplary compositions can be used to treat or prevent cancer, septicemia, obesity, insulin resistance, proliferative diseases such as psoriasis, cerebrovascular diseases, microvascular disease, hypertension, dementia, osteoporosis, arthritis, asthma, heart failure, arrhythmia, angina. Compositions of the invention can also be used as a hormone replacement therapy. In addition, compositions of the invention can be used to treat, prevent or reverse the progression of atherosclerosis, Alzheimer's disease, osteoporosis, or osteopenia; to reduce inflammatory markers; to reduce C-reactive protein; to prevent or treat low grade vascular inflammation, stroke, dementia, coronary heart disease, or stable or unstable angina; for primary and/or secondary prevention of myocardial infarction; for primary prevention of coronary events; for secondary prevention of cardiovascular events; to treat or prevent peripheral vascular disease, peripheral arterial disease, or acute vascular syndromes; to reduce the risk of undergoing a myocardial revascularization procedure; to treat or prevent microvascular diseases (such as nephropathy, neuropathy, retinopathy and nephrotic syndrome), hypertension, Type I and 2 diabetes and related diseases, hyperglycemia, hyperinsulinemia, malignant lesions, premalignant lesions, gastrointestinal malignancies, liposarcomas and epithelial tumors, or proliferative diseases such as psoriasis; to improve coagulation homeostasis and/or endothelial function; and to prevent or treat all forms of cerebrovascular diseases.

Exemplary compositions can be used for the topical applications in wound healing for prevention of scarring.

Exemplary compositions can be used in the treatment or prevention of inflammatory diseases, septic shock and the vascular damage associated with infections and for the treatment of blood and blood products used in dialysis, blood storage in the fluid phase, especially ex vivo platelet aggregation. The compounds in the present invention can also be used in combination with prothrombolytic, fibrinolytic and anticoagulant agents. The present compositions can also be added to human plasma during the analysis of blood chemistry in hospital settings to determine the fibrinolytic capacity thereof.

Exemplary compositions can also be used to treat or prevent cancer including, but not limited to, breast and ovarian cancer, and as imaging agents for the identification of metastatic cancers.

The terms “treat” and “treating,” as used herein, refer to partially or completely alleviating, inhibiting, ameliorating and/or relieving a condition from which a patient is suspected to suffer.

The present invention provides compositions comprising one or more active ingredient. The term “active ingredient” refers to a compound of Formula I and/or pharmaceutically acceptable salt or ester form thereof that is an inhibitor of PAI-1.

In order for a drug administered orally to elicit a therapeutic effect, it typically dissolves into the gastrointestinal fluids and is absorbed through the gastrointestinal wall. The present inventors have discovered that despite the relatively low solubility and bioavailability of the active ingredients disclosed herein, they can be formulated in a dosage form that can be used to dissolve the compound in the body in a way that it can be absorbed through the gastrointestinal wall. In particular, the present inventors have discovered that compositions that combine a surfactant with the active agent can effectively deliver the active ingredient to a patient. In exemplary compositions, the surfactant is distributed in the dosage form by dry blending. In certain exemplary compositions, there will be about 0.2 parts of surfactant per part active ingredient.

The compositions of the present invention can comprise the active compound in any convenient percentage and part in relation to the other ingredients. For use in the present invention, percentages and parts are expressed as part by weight or percentage by weight, unless otherwise noted.

The compounds of Formula I of the present invention can be present in a composition, e.g., oral dosage form, in the form of particles. In some embodiments, these compounds will be in the form of particles having a mean diameter from about 100 to about 400 microns in size. It will be understood that the compounds can be in the form of particles having a mean diameter smaller than about 100 microns or greater than about 400 microns in size.

In some embodiments, the compounds of Formula I of the present invention can be provided in micronized form. For purposes of the present invention, a compound in micronized form is in the form of particles having a mean diameter of no more than about 20 microns. Methods of micronization or particle size reduction are known and are thus not described herein in detail.

Exemplary compositions of the present invention can comprise the active ingredient, for example, 1-Benzyl-3-pentyl-2-[6-(1H-tetrazol-5-ylmethoxy)-2-naphthyl]-1H-indole and/or a pharmaceutically acceptable salt thereof.

In certain exemplary embodiments, a composition of the present invention comprises the active ingredient and at least one surfactant. In certain embodiments of the present invention, there will be about 0.2 parts of surfactant per part active ingredient. The range of surfactant is preferably from about 0.1% to about 10%, more preferably from about 0.3% to about 3.5% surfactant.

The surfactants can be ionic, non-ionic, amphoteric, or cationic. They include, but are not limited to, polyoxyethylene compounds including polyoxyethylene sorbitan fatty acid esters, polyoxyethylene stearates, and glycerol monostearate; polyethylene glycol ethers; saturated polyglycolized glycerides; medium chain monoglycerides including those wherein the chain length is from 6 to 10 carbon atoms, including for example, glyceryl monocaprylate, glyceryl monocaproate, glyceryl caprylate/caprate and a mixture of polyoxyethylene glyceryl caprylate and polyoxyethylene glyceryl caproate; d-α-tocopheryl polyethylene glycol succinate; polyethylene/propylene glycol copolymers; block copolymers of ethylene oxide and propylene oxide; polyoxyl stearates; castor oils derivatives including ethoxylated castor oil such as polyethoxylated (60) hydrogenated castor oil (Cremophor® EL); ethoxylated hydroxystearic acids; lecithins; sulfates including alkyl sulfates and sulfuric acid esters; bile salts,such as cholic acid, deoxy cholic acid, sodium cholate, sodium taurocholate and sodium deoxycholate; carboxylates, such as alkyl carboxylates and alkyl ether carboxylates; docusate salts including the sodium salt thereof; lactylates such as acyl lactylates; N-acyl sarcosinate; carbonates such as polyvalent alkyl carbonate; glutamates such as N-acyl glutamate; ethoxylated amides; alkanolamides; amine oxides; polyoxypropylene compounds; alcohols including propoxylated alcohols and lanolin alcohols; ethoxylated/propoxylated block polymers; simethicone; and esters including, ethoxylated ester, propoxylated esters, fatty acid esters of polyhydric esters, polyglycerol fatty acid esters, sorbitan esters, sucrose esters, glucose (dextrose esters); diethylene glycol esters, propylene glycol esters, fatty acid esters of polyethylene glycols and medium chain fatty acid esters including medium chain length triglycerides, such as a mixture of glyceryl tricaprate and glyceryl tricaprilate; acacia; benzalkonium chloride; cholesterol; and emulsifying wax; and combinations thereof. Additional surfactants can be found in The Handbook of Pharmaceutical Excipients, 2nd Ed., (The Pharmaceutical Press, London and American Pharmaceutical Association (1994)), a common text in the field, which is hereby incorporated by reference in its entirety.

The surfactant is preferably an alkyl sulfate, a poloxamer (e.g., a polyalkylene glycol such as polyethylene or polypropylene glycol), polyethylene glycol, di-fatty acid ester of polyethylene glycols, or a polyoxyalkylene sorbitan ester (e.g., polyoxyethylene sorbitan ester Tween®).

For use herein, the term “poloxamer” refers to a series of non-ionic surfactants that are block copolymers of ethylene oxide and propylene oxide also known as poly(oxyethylene)-poly(oxypropylene) block copolymers.

The polyoxyethylene sorbitan esters (polysorbates) are non-ionic surfactants (detergents) that can comprise a mixture of fatty acids. Commercially available examples are polyoxyethylene (20) sorbitan monolaurate (such as Tween® 20), polyoxyethylene (40) sorbitan monopalmitate (such as Tween® 40), polyoxyethylene (80) sorbitan monooleate (such as Tween® 80) and sorbitan monolaurate (such as Span® 20). Exemplary polyoxyethylene sorbitan fatty acid esters are polyoxyethylene (80) sorbitan monooleate (in particular, Tween® 80).

The alkyl sulfates preferably have from about 8 to about 16 carbon atoms. Commercially available examples are sodium lauryl sulfate or sodium dodecyl sulfate.

The di-fatty acid esters of polyethylene glycols include, for example, saturated polyglycolized glyceride esters including Gelucire®, available from Gattefossé, Saint-Priest, France.

Polyethylene glycols or “PEG”, refer to a liquid or solid polymer of the general formula H(OCH₂CH₂)ₙOH, wherein n is at least 4. The preferred PEG has an average molecular weight of from about 200 to about 5000 Daltons, with a more preferred PEG from about 300 to about 2000 Daltons and a most preferred PEG from about 300 to about 1500 Daltons. Commercially available PEG materials include PEG-200, PEG-300, PEG-400, PEG-540, PEG-600, PEG-800, PEG-1000 and PEG-1450. All are commercially available from, for example, from Union Carbide Corporation in pharmaceutical grades.

In addition to the active ingredient and surfactant, the compositions of the present invention can comprise a number of other excipients including for example, binders, lubricants, diluents, glidants, disintegrants, and combinations thereof.

Disintegrants can also be included in the compositions of the present invention. Disintegrants can be added to the compositions in order to help the capsules disintegrate when they are placed in a liquid environment and so release the active ingredient. The disintegration properties are, mostly, based upon the ability of the disintegrant to swell in the presence of a fluid, such as water or gastric juice. This swelling disrupts the continuity of the capsule structure and thus, allows the different components to enter into solution or into suspension. Disintegrants for use in the present invention include, but are not limited to, starch, starch derivatives, cellulose, cellulose derivatives, alginic acid, alginic acid derivatives, casein, casein derivatives and/or a water-insoluble polyvinylpyrrolidone(crosspolyvidone), and mixtures thereof. The starch is preferably a corn or a potato starch and the starch derivative is preferably a modified starch such as starch glycolate and starch glycolate salts, (e.g., the sodium salt of starch glycolate). The cellulose is preferably carboxymethyl cellulose and/or calcium-and/or sodium carboxymethyl cellulose, in a cross-linked form. In a particularly preferred embodiment of the present invention, a disintegrant is present in the composition at a concentration from about 1% by weight to about 50% by weight, preferably from about 3% by weight to about 20% by weight, even more preferably at about 5% by weight.

Glidants can also be included in the compositions of the present invention. Glidants are substances that are generally used to improve the flow characteristics of granulations and powders by reducing interparticulate friction. Glidants for use in the present invention include, for example, silicon dioxide (e.g., colloidal silicon dioxide), silica gel, asbestos free talc, sodium aluminosilicate, calcium silicate, powdered cellulose, microcrystalline cellulose, sodium benzoate, calcium carbonate, magnesium carbonate, metallic stearates, calcium stearate, magnesium stearate, zinc stearate, syloid, stearowet C, magnesium lauryl sulfate, magnesium oxide, and mixtures thereof. Silicon dioxide is obtained by insolubilizing dissolved silica in sodium silicate solution. When obtained by the addition of sodium silicate to a mineral acid, the product is termed silica gel. When obtained by the destabilization of a solution of sodium silicate in such a manner as to yield very fine particles, the product is termed precipitated silica. In a particularly preferred embodiment of the present invention, the glidant is present in the composition at a concentration from about 0.1% by weight to about 5% by weight, preferably from about 0.3% by weight to about 3% by weight, even more preferably at about 0.5% by weight or 2% by weight depending on the amount of active ingredient in the composition.

The compositions of the invention additionally can include any of a variety of materials that confer beneficial properties to the composition. Such materials include, for example, solubility modifiers such as fillers, lubricants, antioxidants, pH modifiers, chelating agents, binders, stabilizers, excipients including water soluble excipients such as sugars, and water dispersing excipients.

Exemplary lubricants include, for example, magnesium stearate, stearic acid, talc, sodium stearyl fumarate, and mixtures thereof. When a lubricant is present in the composition, the range of lubricant is typically from about, for example, 0.1% to about 5% by weight, more preferably from about 0.5 to about 2%.

Exemplary fillers for use in the invention include, for example, microcrystalline cellulose (e.g., silicified microcrystalline cellulose), carboxymethyl cellulose, lactose, calcium carbonate, calcium phosphate, maltodextrin, sugar alcohols, such as mannitol, dextrose, sucrose, fructose, maltose, and mixtures thereof. The range of filler is typically from about, for example, 15% to about 98% by weight.

In some embodiments, the compositions will be substantially free of acid excipients. In others, the composition will comprise at least one acid excipient, such as, for example, an organic acid. For uses herein, the term “organic acid” encompasses any acid that can be safely ingested by a mammal. Examples of organic acids suitable for use in the present invention include, but are not limited to, tartaric acid, malic acid, fumaric acid, aspartic acid, glutamic acid, glycine hydrochloride, adipic acid, succinic acid, ascorbic acid, oleic acid or citric acid. Preferred organic acids are citric acid or polyfunctional organic acid. The range of organic acid in the composition is preferably 1% or smaller.

Nonlimiting examples of stabilizers include antioxidants such as BHA, BHT, ascorbic acids, tocopherols, and the like. Nonlimiting examples of suitable metal chelators include EDTA, citric acid and the like. Nonlimiting examples of pH modifiers include citric acid, fumaric acid, and the like. Nonlimiting examples of binders include starches, PVP (polyvinylpyrrolidone), HPMC (hydroxypropyl methyl celluloses), HPC (hydroxypropyl cellulose) and the like.

The compositions of the present invention can contain the active compound in any convenient percentage and part in relation to the other ingredients. Typically, the composition comprises active ingredient in percentage of from about 0.05% to about 25%, more preferably from about 1% to about 20%. In certain preferred embodiments, the percentage of active ingredient will be from about 1 to about 2% or from about 10 to about 20%.

The compositions of the present invention can be formed, for example, by dry granulation, direct compression, wet granulation or dry blending. Dry granulation generally includes mixing the ingredients, slugging the ingredients, dry screening, lubricating and finally compressing the ingredients. In direct compression, the powdered material(s) to be included in the solid dosage form is compressed directly without modifying the physical nature of the material itself. The wet granulation procedure includes mixing the powders to be incorporated into the dosage form in, e.g., a twin shell blender or double-cone blender, and thereafter adding solutions of a binding agent to the mixed powders to obtain a granulation. The damp mass is screened, e.g., in a mesh screen and then dried. In dry blending, the ingredients are simply blended together. In some embodiments, the compositions are prepared by roller compaction. For example, capsules or tablets can be prepared by granulation followed by milling. In some embodiments, the active ingredient and/or one or more additional excipients are granulated and then milled. The milled granules can then mixed with additional excipients that are not granulated or milled

The compositions of the present invention can be, for example, in the form of coated or uncoated pellets, spheres, capsules (e.g., hard or soft gelatin capsules), powder, or tablets.

Thus, in accordance with the present invention there are provided immediate release dosage forms, including oral and non-oral immediate release dosage compositions. Accordingly, the present invention includes each of the numerous technologies that exist for immediate release non-oral dosage composition. Delivery of active compound in accordance with the present invention can be via mucosal, vaginal, rectal, ocular, transdermal, intrauterine, routes and the like.

The present invention therefore provides, inter alia, compositions for substituted naphthyl indole derivatives of the present invention and methods for immediate delivery of substituted naphthyl indole derivatives In some embodiments, administration of the composition will be once every 24 hours, once every 12 hours, or once every 6 hours. Preferably, the compositions will be orally administered.

In certain embodiments, the compositions will comprise about 10 to 15% by weight of the active ingredient (i.e., a compound of formula I such as 1-Benzyl-3-pentyl-2-[6-(1H-tetrazol-5-ylmethoxy)-2-naphthyl]-1H-indole), about 3% to 5% by weight of a surfactant (i.e., sodium lauryl sulfate), about 5% to 10% by weight of a disintegrant (i.e., cross-linked sodium carboxymethylcellulose), about 60-85% by weight of a filler (i.e., microcrystalline cellulose), about 0 to 5% by weight of a glidant (i.e., silicon dioxide), and about 0 to 5% by weight of a lubricant (i.e., magnesium stearate). In certain exemplary embodiments, there will be about 0.2 parts of surfactant per part active ingredient

In certain embodiments, the compositions will comprise about 17% by weight of the active ingredient (i.e., a compound of formula I such as 1-Benzyl-3-pentyl-2-[6-(1H-tetrazol-5-ylmethoxy)-2-naphthyl]-1H-indole), about 3% by weight of a surfactant (i.e., sodium lauryl sulfate), about 5% by weight of a disintegrant (i.e., cross-linked sodium carboxymethylcellulose), about 15% by weight of a filler (i.e., microcrystalline cellulose,), about 56% by weight of a second filler (i.e., mannitol) about 2% by weight of a glidant (i.e., silicon dioxide), and about 2% by weight of a lubricant (i.e., magnesium stearate). In certain exemplary embodiments, there will be about 0.2 parts of surfactant per part active ingredient

In certain embodiments, the compositions will comprise about 1 to 3% by weight of the active ingredient (i.e., a compound of formula I such as 1-Benzyl-3-pentyl-2-[6-(1H-tetrazol-5-ylmethoxy)-2-naphthyl]-1H-indole), about 0.1% to 0.5% by weight of a surfactant (i.e., sodium lauryl sulfate), about 5% to 10% by weight of a disintegrant (i.e., cross-linked sodium carboxymethylcellulose), about 85-95% by weight of a filler (i.e., microcrystalline cellulose), about 0 to 1% by weight of a glidant (i.e., silicon dioxide), and about 0 to 1% by weight of a lubricant (i.e., magnesium stearate). In certain exemplary embodiments, there will be about 0.2 parts of surfactant per part active ingredient

In certain embodiments, the compositions will comprise about 2% by weight of the active ingredient (i.e., a compound of formula I such as 1-Benzyl-3-pentyl-2-[6-(1H-tetrazol-5-ylmethoxy)-2-naphthyl]-1H-indole), about 0.3% by weight of a surfactant (i.e., sodium lauryl sulfate), about 5% by weight of a disintegrant (i.e., cross-linked sodium carboxymethylcellulose), about 15% by weight of a filler (i.e., microcrystalline cellulose,), about 77% by weight of a second filler (i.e., mannitol) about 0.5% by weight of a glidant (i.e., silicon dioxide), and about 0.5% by weight of a lubricant (i.e., magnesium stearate). In certain exemplary embodiments, there will be about 0.2 parts of surfactant per part active ingredient.

The “plasma drug concentration” or “plasma concentration” refers to the concentration of drug in the blood plasma of a subject, generally expressed as mass per unit volume, typically nanograms per milliliter. The plasma drug concentration at any time following drug administration is referenced as Cₜᵢₘₑ, as in C₉ₕ or C_{24 h}.

Persons of skill in the art appreciate that plasma drug concentrations obtained in individual subjects will vary due to interpatient variability in the many parameters affecting drug absorption, distribution, metabolism and excretion. For this reason, unless otherwise indicated, mean values obtained from groups of subjects are used herein for purposes of comparing plasma drug concentration data and for analyzing relationships between in vitro dosage form dissolution rates and in vivo plasma drug concentrations.

In certain exemplary embodiments, the compositions of the present invention exhibit an in vitro dissolution profile in which about 90% or greater of the active ingredient is released after 45 minutes of measurement. In certain embodiments, a composition comprising 1 mg of active ingredient releases on average about 97% of the active ingredient after 45 minutes measurement, a composition comprising 5 mg of active ingredient releases on average about 94% of the active ingredient after 45 minutes measurement, and a composition comprising 25 mg of active ingredient releases on average about 100% of the active ingredient after 45 minutes measurement.

The dissolution is determined as directed in the USP, using Apparatus 2 (paddles), at 50 rpm, in 900 mL or 500 mL of 0.1% Tween 80 in 0.05 M sodium dihydrogen phosphate buffer pH 6.0 at 37° C. A filtered sample of the dissolution medium is taken at the time(s) specified.

For 25 and 5 mg capsules the uv-visible spectrum of the clear solution is recorded over the range 200 nm to 500 nm against an appropriate capsule blank solution. The absorbance at the wavelength of maximum absorbance at about 305 nm is determined with respect to this baseline. The amount of active ingredient dissolved is determined by comparing this absorbance to that of a standard solution prepared concomitantly. The range of 490-500 nm is subtracted as a background correction

For 1 mg capsules the amount of active ingredient dissolved is determined by chromatographing the sample on a reversed-phase high performance liquid chromatography column. The concentration of active ingredient in each sample is determined by comparing the peak responses of the sample chromatogram with the peak responses of the standard chromatograms obtained concomitantly.

As described herein, the present invention provides compositions comprising one or more compound of Formula I. A subset of the compounds are those of the Formula I: 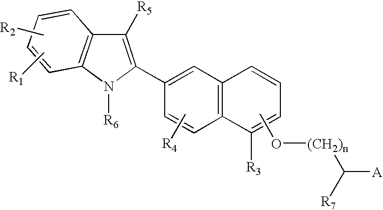
wherein R₁, R₂, R₃, R₄, R₅, R₆, R₇, A, n, and R₈ are as defined above, or a pharmaceutically acceptable salt or ester form thereof.

A further subset of the compounds of this invention comprises those having the Formula I: 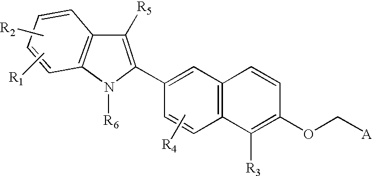
wherein:

R₁, R₂, and R₃, are each, independently, hydrogen, alkyl of 1-3 carbons, cycloalkyl of 3-5 carbons, alkanoyl of 1-3 carbons, halogen, hydroxy, aryl optionally substituted with from 1 to 3 groups selected from R₆, perfluoroalkyl of 1-3 carbons, alkoxy of 1-3 carbons, amino, alkylamino of 1-3 carbons, dialkylamino of 1-3 carbons per alkyl group, perfluoroalkoxy of 1-3 carbons;

R₄ is hydrogen, alkyl of 1-6 carbons, perfluoroalkyl of 1-6 carbons, aryl substituted with R₆, alkanoyl of 1-6 carbons, aroyl optionally substituted with from 1 to 3 groups selected from R₆;

R₅ is hydrogen, alkyl of 1-6 carbons, alkylaryl, benzyl optionally substituted with from 1 to 3 groups selected from R₆, alkanoyl of 1-6 carbons, aroyl substituted with R₆;

A is COOH or tetrazole;

R₆ is hydrogen, alkyl of 1-3 carbons, cycloalkyl of 3-5 carbons, —CH₂-cycloalkyl of 3-5 carbons, alkanoyl of 1-3 carbons, halogen, hydroxy, perfluoroalkyl of 1-3 carbons, alkoxy of 1-3 carbons, amino, alkylamino of 1-3 carbons, dialkylamino of 1-3 carbons, perfluoroalkoxy of 1-3 carbons; or a pharmaceutically acceptable salt or ester form thereof.

As used herein, “alkyl” refers to an aliphatic hydrocarbon chain and includes straight and branched chains such as methyl, ethyl, n-propyl, isopropyl, n-butyl, isobutyl, sec-butyl, t-butyl, n-pentyl, isopentyl, neo-pentyl, n-hexyl, and isohexyl. Exemplary alkyl groups have from 1 to 6 carbon atoms. Halogen refers to bromine, chlorine, fluorine, and iodine.

Ester forms of the compounds of Formula I include the pharmaceutically acceptable ester forms known in the art for the acid groups of Formula I, above. These esters include straight chain alkyl esters having from 1 to 6 carbon atoms or branched chain alkyl groups containing 3 or 6 carbon atoms, including methyl, ethyl, propyl, butyl, 2-methylpropyl and 1,1-dimethylethyl esters. Other non-limiting examples of esters useful with this invention include those wherein A is a carboxylic acid and the ester form has the formula —COOR₁₄ wherein R₁₄ is selected from the formulae: 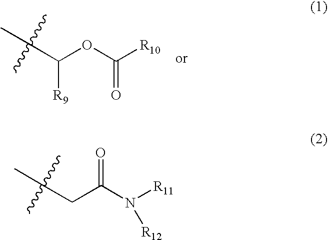
wherein R₉, R₁₀, R₁₁ and R₁₂ are independently selected from the group consisting of hydrogen, alkyl of from 1 to 10 carbon atoms, aryl of 6 to 12 carbon atoms, arylalkyl of from 6 to 12 carbon atoms, heteroaryl and alkylheteroaryl wherein the heteroaryl ring is bound by an alkyl chain of from 1 to 6 carbon atoms.

Among the preferred ester forms of the compounds herein include but not limited to C₁-C₆ alkyl esters, C₃-C₆ branched alkyl esters, benzyl esters, and the like.

The substituents of A as defined herein are acidic groups, including acid mimics or mimetics. Carboxylic acid mimics or mimetics are described in R. Silverman, The Organic Chemistry of Drug Design and Drug Action, Academic Press (1992), the contents of which are incorporated herein by reference. Non-limiting examples of acid mimics include tetrazole, SO₃H, PO₃H₂, tetronic acid, or groups having the formula: 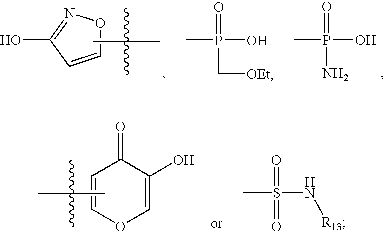
wherein R₁₃ is C₁-C₆ alkyl, C₂-C₆ alkenyl, C₃-C₆ cycloalkyl, —CH₂—(C₃-C₆ cycloalkenyl, —CH₂—(C₃-C₆ cycloalkenyl), optionally substituted aryl or heteroaryl groups or optionally substituted —C₁-C₆ alkyl-aryl or —C₁-C₆ alkyl-heteroaryl, with the aryl and heteroaryl groups and their optional substitution as defined herein.

As used herein, “aryl” refers to an unsaturated aromatic carbocyclic group of from 6 to 14 carbon atoms having a single ring (e.g., phenyl) or multiple condensed (fused) rings (e.g., naphthyl or anthryl). Preferred aryl groups include phenyl, naphthyl and the like. As used herein, “heteroaryl” refers to a monocyclic or bicyclic aromatic group of from 1 to 9 carbon atoms and 1 to 4 heteroatoms selected from oxygen, nitrogen and sulfur within at least one ring (if there is more than one ring). Such heteroaryl groups can have a single ring, such as pyridyl, pyrrolyl or furyl groups, or multiple condensed rings, such as indolyl, indolizinyl, benzofuranyl or benzothienyl groups. Preferred heteroaryls include pyridyl, pyrrolyl and furyl. It will be understood that the definitions of aryl and heteroaryl also refer to those portions of any aroyl or heteroaroyl groups described herein.

Unless otherwise limited by the definition for the aryl or heteroaryl groups herein, such groups can optionally be substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl of 1 to 6 carbon atoms, substituted alkoxy of 1 to 6 carbon atoms, substituted alkenyl of 2 to 6 carbon atoms, substituted alkynyl of 2 to 6 carbon atoms, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. Substituents on the alkyl, alkenyl, alkynyl, thioalkoxy and alkoxy groups mentioned above include halogens, CN, OH, and amino groups. Preferred substituents on the aryl groups herein include alkyl, alkoxy, halo, cyano, nitro, trihalomethyl, and thioalkoxy.

The term “acyl”, employed alone or in combination with other terms, is defined herein as, unless otherwise stated, either an alkyl, arylalkyl, heteroarylalkyl, (C₂-C₁₀) straight chain, or (C₄-C₁₁) branched-chain monovalent hydrocarbon moiety; wherein the carbon atom, covalently linked to the defined chemical structure, is oxidized to the carbonyl oxidation state. Such hydrocarbon moieties may be mono or polyunsaturated, and may exist in the E or Z configurations. Examples of acyl moieties include, but are not limited to, chemical groups such as acetyl, propionyl, butyryl, 3,3-dimethylbutyryl, trifluoroacetyl, pivaloyl, hexanoyl, hexenoyl, decanoyl, benzoyl, nicotinyl, isonicotinyl, and homologs, isomers, and the like.

Pharmaceutically acceptable salts of compounds of Formula I containing a basic group, such as amino or alkylamino groups, can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. Salts can also be formed from organic and inorganic bases, preferably alkali metal salts, for example, sodium, lithium, or potassium.

Other useful salt forms of these compounds include those formed with pharmaceutically acceptable inorganic and organic bases known in the art. Salt forms prepared using inorganic bases include hydroxides, carbonates or bicarbonates of the therapeutically acceptable alkali metals or alkaline earth metals, such as sodium potassium, magnesium, calcium and the like. Acceptable organic bases include amines, such as benzylamine, mono-, di- and trialkylamines, preferably those having alkyl groups of from 1 to 6 carbon atoms, more preferably 1 to 3 carbon atoms, such as methylamine, dimethylamine, trimethylamine, ethylamine, diethylamine, triethylamine, mono-, di-, and triethanolamine. Also useful are alkylene diamines containing up to 6 carbon atoms, such as hexamethylenediamine; cyclic saturated or unsaturated bases containing up to 6 carbon atoms, including pyrrolidine, peperidine, morpholine, piperazine and their N-alkyl and N-hydroxyalkyl derivatives, such as N-methyl-morpholine and N-(2-hyroxyethyl)-piperidine, or pyridine. Quaternary salts can also be formed, such as tetralkyl forms, such as tetramethyl forms, alkyl-alkanol forms, such as methyl-triethanol or trimethyl-monoethanol forms, and cyclic ammonium salt forms, such as N-methylpyridinium, N-methyl-N-(2-hydroxyethyl)-morpholinium, N,N-di-methylmorpholinium, N-mehtyl-N-(2-hydroxyethyl)-morpholinium, or N,N-dimethyl-piperidinium salt forms. These salt forms can be prepared using the acidic compound(s) of Formula I and procedures known in the art.

The compounds of Formula I can contain an asymmetric carbon atom or sulfoxide moiety and some of the compounds of this invention can contain one or more asymmetric centers and can thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof.

Compounds of Formula I can be prepared by those skilled in the art of organic synthesis employing known methods that utilize readily available reagents and starting materials, see, for example, U.S. Pat. No. 6,800,654, the disclosure of which is incorporated herein by reference in its entirety for all purposes.

The present invention provides compositions comprising compounds of Formula I and in particular, compositions comprising 1-Benzyl-3-pentyl-2-[6-(1H-tetrazol-5-ylmethoxy)-2-naphthyl]-1H-indole or a pharmaceutically acceptable salt thereof; 6-(1-Benzyl-3-pentyl-1H-indol-2-yl)-1-bromo-2-naphthyl 1H-tetrazol-5-ylmethyl ether or 1-Benzyl-2-[5-bromo-6-(1H-tetrazol-5-ylmethoxy)-2-naphthyl]-3-pentyl-1H-indole or a pharmaceutically acceptable salt thereof; 1-Methyl-3-pentyl-2-[6-(1H-tetrazol-5-ylmethoxy)-2-naphthyl]-1H-indole or a pharmaceutically acceptable salt thereof; 2-[5-Bromo-6-(1H-tetrazol-5-ylmethoxy)-2-naphthyl]-1-methyl-3-pentyl-1H-indole or 1-Bromo-6-(1-methyl-3-pentyl-1H-indol-2-yl)-2-napthyl 1H-tetrazol-5-ylmethyl ether or a pharmaceutically acceptable salt thereof; 1-Acetyl-3-pentyl-2-[6-(1H-tetrazol-5-ylmethoxy)-2-naphthyl]-1H-indole or a pharmaceutically acceptable salt thereof; 1-Acetyl-2-[5-bromo-6-(1H-tetrazol-5-ylmethoxy)-2-naphthyl]-3-pentyl-1H-indole or a pharmaceutically acceptable salt thereof; 3-Pentyl-2-[6-(1H-tetrazol-5-ylmethoxy)-2-naphthyl]-1-[2-(trifluoromethyl)benzyl]-1H-indole or a pharmaceutically acceptable salt thereof; 2-[5-Bromo-6-(1H-tetrazol-5-ylmethoxy)-2-naphthyl]-3-pentyl-1-[2-(trifluoromethyl)benzyl]-1H-indole or a pharmaceutically acceptable salt thereof; 1-(4-tert-Butylbenzyl)-3-pentyl-2-[6-(1H-tetrazol-5-ylmethoxy)-2-naphthyl]-1H-indole or a pharmaceutically acceptable salt thereof; 2-[5-Bromo-6-(1H-tetrazol-5-ylmethoxy)-2-naphthyl]-1-(4-tert-butylbenzyl)-3-pentyl-1H-indole or a pharmaceutically acceptable salt thereof; {[1-Bromo-6-(1-methyl-3-pentyl-1H-indol-2-yl)-2-naphthyl]oxy}acetic acid or a pharmaceutically acceptable salt thereof, or combinations thereof.

### EXAMPLES

### Example 1

Representative Oral Dosage Forms:

High Strength Blend

| Ingredient | % weight/weight | mg/cap |
|---|---|---|
| Compound of Formula I | 16.67 | 25.00 |
| Surfactant (e.g., SLS) | 3.33 | 5.00 |
| Disintegrant (e.g., cross- | 5.00 | 7.5 |
| linked sodium | | |
| carboxymethylcellulose, | | |
| such as AcDiSol) | | |
| Filler (e.g., | 15.00 | 22.50 |
| microcrystalline cellulose, | | |
| such as Avicel PH200) | | |
| Glidant (e.g., silicon | 2.00 | 3.00 |
| dioxide such as Aerosil | | |
| 200) | | |
| Filler (e.g., Mannitol) | 56.00 | 84.00 |
| Lubricant (e.g., Magnesium | 2.00 | 3.00 |
| Stearate) | | |
| Total/Fill in HPMC Cap | 100.00 | 150.00 |

Low Strength Blend

| Ingredient | % weight/weight | mg/cap | mg/cap |
|---|---|---|---|
| Compound of Formula I | 1.67 | 1 | 5.0 |
| Surfactant (e.g., SLS) | 0.33 | 0.2 | 1.0 |
| Disintegrant (e.g., | 5.00 | 3.0 | 15.0 |
| cross-linked sodium | | | |
| carboxymethylcellulose, | | | |
| such as AcDiSol) | | | |
| Filler (e.g., | 15.00 | 9.0 | 45.0 |
| microcrystalline | | | |
| cellulose, such as | | | |
| Avicel PH200) | | | |
| Glidant (e.g., silicon | 0.5 | 0.3 | 1.5 |
| dioxide such as Aerosil | | | |
| 200) | | | |
| Filler (e.g., Mannitol) | 77.00 | 46.2 | 231.0 |
| Lubricant (e.g., | 0.5 | 0.3 | 1.5 |
| Magnesium Stearate) | | | |
| Total/Fill in HPMC | 100.00 | 60.00 | 300.00 |
| Cap | | | |

Representative Manufacturing for Low Strength Blend:

Blend the ingredinets by a three stage geometric dilution in the following order.

Stage 1 (1:3)

| | |
|---|---|
| Active ingredient | 6.74 g |
| Glidant | 2.00 g |
| Surfactant | 1.32 g |
| Filler | 16.90 g |

Screen through 500 um into Turbula mixer, mix for 10 minutes, screen through 500 um into Turbula mixer.

Stage 2 (1:3)

| | |
|---|---|
| Stage 1 | 26.96 g |
| Disinegrant | 20.0 g |
| Filler | 43.10 g |

Screen through 500 um into Turbula mixer, mix for 10 minutes, screen through 500 um into Turbula mixer.

Stage 3 (1:3.7)

| | |
|---|---|
| Stage 2 | 107.84 g |
| Filler | 290.16 g |

Screen through 500 um into Turbula mixer, mix for 10 minutes, screen through 500 um into Turbula mixer.

What is claimed:

1. A composition comprising:
about 1% to about 90% by weight of an active ingredient of Formula I: 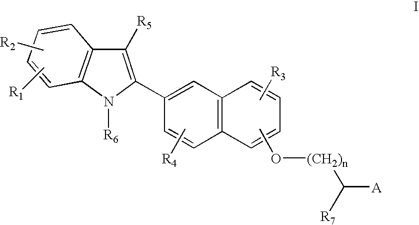
wherein:
R₁, R₂, R₃, and R₄ are each, independently, hydrogen, alkyl of 1-6 carbons, cycloalkyl of 3-5 carbon atoms, —CH₂-cycloalkyl of 3-5 carbon atoms, alkanoyl of 1-6 carbons, halogen, hydroxy, aryl optionally substituted with from 1 to 3 groups selected from R₈, perfluoroalkyl of 1-6 carbons, alkoxy of 1-6 carbons, amino, alkylamino of 1-6 carbons, dialkylamino of 1-6 carbons, or perfluoroalkoxy of 1-6 carbons;
R₅ is hydrogen, alkyl of 1-6 carbons, perfluoroalkyl of 1-6 carbons, aryl substituted with R₈, alkanoyl of 1-6 carbons, or aroyl optionally substituted with from 1 to 3 groups selected from R₈;
R₆ is hydrogen, alkyl of 1-6 carbons, alkylaryl, benzyl substituted with R₈, alkanoyl of 1-6 carbons, or aroyl optionally substituted with from 1 to 3 groups selected from R₈;
R₇ is hydrogen, alkyl of 1-6 carbons, alkylaryl, or aryl optionally substituted with from 1 to 3 groups selected from R₈;
n is an integer of 0-6;
A is COOH, or an acid mimic; and
R₈ is hydrogen, alkyl of 1-6 carbons, cycloalkyl of 3-5 carbons, —CH₂-cycloalkyl of 3-5 carbon atoms, alkanoyl of 1-6 carbons, halogen, hydroxy, perfluoroalkyl of 1-6 carbons, alkoxy of 1-6 carbons, amino, alkylamino of 1-6 carbons, dialkylamino of 1-6 carbons, or perfluoroalkoxy of 1-6 carbons,
or a pharmaceutically acceptable salt or ester form thereof; and
about 0.1% to about 10% by weight of a surfactant.

2. The composition of claim 1 wherein
R₁, R₂, R₃, and R₄ are each, independently, hydrogen, alkyl of 1-3 carbons, cycloalkyl of 3-5 carbon atoms, —CH₂-cycloalkyl of 3-5 carbon atoms, alkanoyl of 1-3 carbons, halogen, hydroxy, aryl optionally substituted with from 1 to 3 groups selected from R₈, perfluoroalkyl of 1-3 carbons, alkoxy of 1-3 carbons, amino, alkylamino of 1-3 carbons, dialkylamino of 1-3 carbons, or perfluoroalkoxy of 1-3 carbons; and
R₈ is hydrogen, alkyl of 1-3 carbons, cycloalkyl of 3-5 carbons, —CH₂-cycloalkyl of 3-5 carbon atoms, alkanoyl of 1-3 carbons, halogen, hydroxy, perfluoroalkyl of 1-3 carbons, alkoxy of 1-3 carbons, amino, alkylamino of 1-3 carbons, dialkylamino of 1-3 carbons, or perfluoroalkoxy of 1-3 carbons.

3. The composition of claim 1 wherein the ratio of active ingredient to surfactant is about 5:1 w/w.

4. The composition of claim 1 wherein said surfactant is an alkyl sulfate, a polyoxyalkylene sorbitan ester, or a poloxamer.

5. The composition of claim 1 wherein said compound of Formula I is 1-Benzyl-3-pentyl-2-[6-(1H-tetrazol-5-ylmethoxy)-2-naphthyl]-1H-indole or a pharmaceutically acceptable salt or ester form thereof.

6. The composition of claim 1 further comprising a disintegrant.

7. The composition of claim 6 wherein said disintegrant is carboxymethyl cellulose or a pharmaceutically acceptable basic salt form thereof; starch glycolate or a pharmaceutically acceptable basic salt form thereof; or mixtures thereof.

8. The composition of claim 7 wherein said disintegrant is the sodium salt of cross-linked carboxymethyl cellulose and said starch glycolate is the sodium salt of starch glycolate.

9. The composition of claim 1 further comprising a glidant.

10. The composition of claim 9 wherein said glidant is silicon dioxide, silica gel, or mixtures thereof.

11. The composition of claim 1 further comprising at least one lubricant and at least one filler.

12. The composition of claim 11 wherein said lubricant is magnesium stearate, stearic acid, talc, sodium stearyl fumarate, or mixtures thereof.

13. The composition of claim 11 wherein said filler is microcrystalline cellulose, lactose, calcium carbonate, calcium phosphate, maltodextrin, dextrose, sucrose, fructose, maltose, mannitol, starch, or mixtures thereof.

14. The composition of claim 1 that comprises from about 1% to about 2% by weight of a compound of Formula I or a pharmaceutically acceptable salt or ester form thereof, and further comprises about 3% to about 20% by weight of a disintegrant and about 0.1% to about 5% by weight of a glidant.

15. The composition of claim 1 that comprises from about 10% to about 20% by weight of a compound of Formula I or a pharmaceutically acceptable salt or ester form thereof, and further comprises about 3% to about 20% by weight of a disintegant and about 0.1% to about 5% by weight of a glidant.

16. The composition of claim 15 wherein said disintegrant is carboxymethyl cellulose; starch glycolate; or a pharmaceutically acceptable basic salt form thereof; and said glidant is silicon dioxide or silica gel.

17. The composition of claim 16 wherein said disintegrant is carboxymethyl cellulose; starch glycolate; or a pharmaceutically acceptable basic salt form thereof; and said glidant is silicon dioxide or silica gel.

18. The composition of claim 1 that is in the form of a capsule.

19. The composition of claim 1 that is in the form of a dry blend.

20. A process comprising mixing a compound of Formula I: 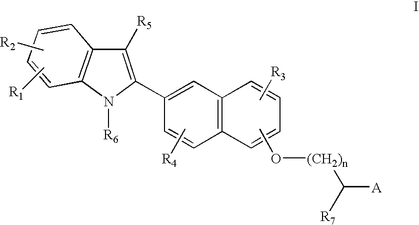
wherein:
R₁, R₂, R₃, and R₄ are each, independently, hydrogen, alkyl of 1-6 carbons, cycloalkyl of 3-5 carbon atoms, —CH₂-cycloalkyl of 3-5 carbon atoms, alkanoyl of 1-6 carbons, halogen, hydroxy, aryl optionally substituted with from 1 to 3 groups selected from R₈, perfluoroalkyl of 1-6 carbons, alkoxy of 1-6 carbons, amino, alkylamino of 1-6 carbons, dialkylamino of 1-6 carbons, or perfluoroalkoxy of 1-6 carbons;
R₅ is hydrogen, alkyl of 1-6 carbons, perfluoroalkyl of 1-6 carbons, aryl substituted with R₈, alkanoyl of 1-6 carbons, or aroyl optionally substituted with from 1 to 3 groups selected from R₈;
R₆ is hydrogen, alkyl of 1-6 carbons, alkylaryl, benzyl substituted with R₈, alkanoyl of 1-6 carbons, or aroyl optionally substituted with from 1 to 3 groups selected from R₈;
R₇ is hydrogen, alkyl of 1-6 carbons, alkylaryl, or aryl optionally substituted with from 1 to 3 groups selected from R₈;
n is an integer of 0-6;
A is COOH, or an acid mimic; and
R₈ is hydrogen, alkyl of 1-6 carbons, cycloalkyl of 3-5 carbons, —CH₂-cycloalkyl of 3-5 carbon atoms, alkanoyl of 1-6 carbons, halogen, hydroxy, perfluoroalkyl of 1-6 carbons, alkoxy of 1-6 carbons, amino, alkylamino of 1-6 carbons, dialkylamino of 1-6 carbons, or perfluoroalkoxy of 1-6 carbons,
or a pharmaceutically acceptable salt or ester form thereof;
with at least one surfactant; at least one disintegrant; and at least one glidant thereby forming a mixture blend thereof.

21. A method for treating or preventing impairment of the fibrinolytic system, thrombosis, atrial fibrillation, pulmonary fibrosis, myocardial ischemia, stroke, thromboembolic complication of surgery, cardiovascular disease, atherosclerotic plaque formation, chronic obstructive pulmonary disease, renal fibrosis, polycystic ovary syndrome, or diabetes in a subject comprising administering to the subject an effective amount of a composition of claim 1.

22. The method of claim 21 wherein the thrombosis is selected from the group consisting of venous thrombosis, arterial thrombosis, cerebral thrombosis, and deep vein thrombosis.

23. The method of claim 20 wherein the cardiovascular disease is caused by noninsulin dependent diabetes mellitus in a subject.
